(19) United States
(12) Patent Application Publication
(10) Pub. No.: US 2023/0015958 A1
(43) Pub. Date: Jan. 19, 2023

(54) PEN NEEDLE SHIELD

(71) Applicant: Embecta Corp., Andover, MA (US)

(72) Inventors: Kamil Szymaniak, New Providence, NJ (US); Paul Murray, Navan Meath (IE); Sergey Grigoryants, Malden, MA (US)

(73) Assignee: Embecta Corp., Andover, MA (US)

(21) Appl. No.: 17/379,286

(22) Filed: Jul. 19, 2021

(51) Int. Cl. A61M 5/32

(52) U.S. Cl. CPC A61M 5/3271; A61M 5/3257; A61M 2005/3254; A61M 2005/3247

(57) ABSTRACT

A pen needle shield assembly comprising a hub fixed to a needle cannula, and a movable shield surrounding the hub, the movable shield comprising an upper portion having an outer diameter, a lower portion having an outer diameter greater than the outer diameter of the upper portion, and a distal opening, wherein the movable shield moves from a first position where the needle cannula is retracted in the movable shield prior to use, to a second position where the needle cannula is extended out of the movable shield through the distal opening for medication delivery and to a third position where the needle cannula is retracted in the movable shield and locked to prevent further use.

### FIELD OF THE INVENTION

The present invention relates to improvements to existing pen needle shields such as a safety pen needle.

### BACKGROUND OF THE INVENTION

Various factors that increase material and labor costs for existing pen needle shields include the number of components, molding equipment and operation and secondary post processing operations. Current patient end shielding assemblies involve spring subassemblies and multiple moving parts. In view of these considerations, an improved pen needle shield is desired.

### SUMMARY OF THE INVENTION

It is an aspect of the present invention to provide a pen needle shield that incorporates two manufactured components in combination with a spring. Typically, commonly used pen needle shields include four or more components. Such a configuration disclosed herein advantageously removes at least one manufactured component from the assembly to provide a minimal number of components, improved cost and reduced assembly time.

It is another aspect of the present invention to design manufactured components to avoid involving any side action during the molding process. Instead, a simple core-cavity mold is used to manufacture the components. Such a configuration advantageously provides simple, moldable parts that reduces molding costs and allows for high cavitation molds.

Finally, it is another aspect of the present invention to provide the above described pen needle shield assembly with an improved assembly process. Specifically, the pen needle shield assembly is assembled in a straight down snap fit motion. Such a configuration simplifies the assembly process and also reduces the assembly time of the pen needle shield.

The foregoing and/or other aspects of the present invention can be achieved by providing a pen needle shield assembly comprising a hub fixed to a needle cannula, and a movable shield surrounding the hub, the movable shield comprising an upper portion having an outer diameter, a lower portion having an outer diameter greater than the outer diameter of the upper portion, and a distal opening, wherein the movable shield moves from a first position where the needle cannula is retracted in the movable shield prior to use, to a second position where the needle cannula is extended out of the movable shield through the distal opening for medication delivery and to a third position where the needle cannula is retracted in the movable shield and locked to prevent further use.

Additional and/or other aspects and advantages of the present invention will be set forth in the description that follows, or will be apparent from the description, or may be learned by practice of the invention.

### BRIEF DESCRIPTION OF THE DRAWINGS

The above aspects and features of the present invention will be more apparent from the description for the exemplary embodiments of the present invention taken with reference to the accompanying drawings, in which:

FIG. 1 illustrates a front perspective view of an exemplary pen needle shield in a first position;

FIG. 2 illustrates a front perspective view of a second exemplary embodiment of a pen needle shield in a first position;

FIG. 3 illustrates a front perspective view of the pen needle shield of FIG. 2 in a third position; and

FIG. 4 illustrates a left perspective view of the pen needle shield of FIG. 2 in the third position;

FIG. 5 illustrates a front perspective view of a hub in the pen needle shield of FIG. 2; and

FIG. 6 illustrates a front perspective view of the hub of FIG. 5 and a spring.

### DETAILED DESCRIPTION OF EXEMPLARY EMBODIMENTS

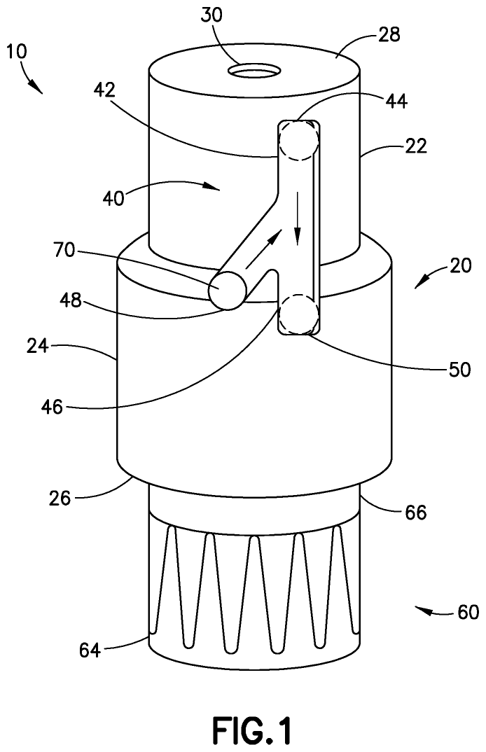

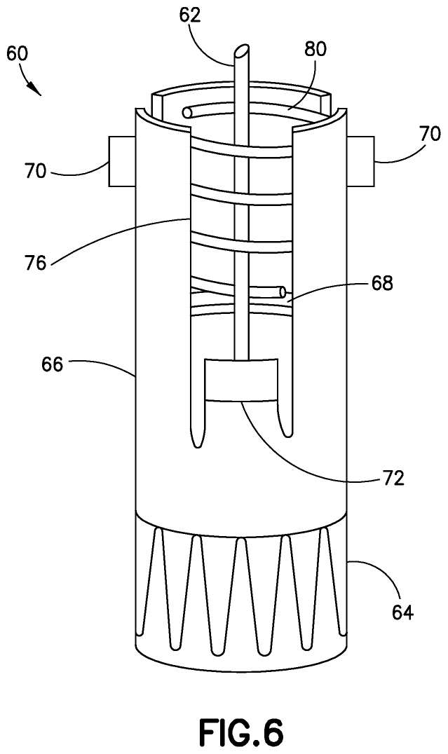

FIG. 1 illustrates a pen needle shield assembly 10 comprising a movable shield 20, a hub 60 and a spring 80 (the spring is illustrated in FIG. 6). The movable shield 20 surrounds a distal portion of the hub 60. The movable shield 20 includes an upper portion 22, a lower portion 24, a proximal end 26 and a distal end 28 having a distal opening 30. The upper portion 22 has an outer diameter that is smaller than an outer diameter of the lower portion 24.

The shape of the movable shield 20 advantageously allows for improved manufacturability. Specifically, the upper portion 22 and the lower portion 24 having different sizes allows for the movable shield 20 to be molded without any side action. Instead, a simple core-cavity mold is used to manufacture the movable shield 20. This allows for steel from the inner core of the mold to shape and mold an upper track 42 of a cam mechanism 40 in the upper portion 22. This also allows for the steel from the outer cavity of the mold to shape and mold a lower track 46 of the cam mechanism 40 in the lower portion 24. As a result, a simple, moldable design is provided where high cavitation molds are possible and mold costs are reduced.

The distal end 28 of the movable shield 20 includes the distal opening 30 so that a needle cannula 62 can be exposed as further described below. The proximal end 26 of the movable shield 20 cooperates with hub 60 to creating a locking mechanism for needle stick prevention. The elements of the locking mechanism are also described below.

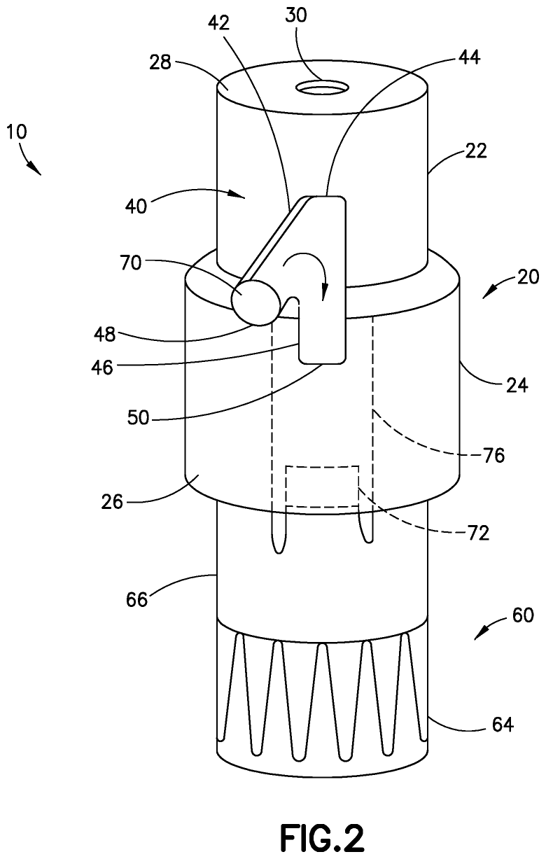

As illustrated in FIG. 1, the movable shield 20 further includes the cam mechanism 40. FIGS. 1 and 2 illustrates two different shapes of the cam mechanism 40 although a variety of sizes and shapes are contemplated by one skilled in the art. The cam mechanism 40 is formed as an opening that extends through an outer surface of the movable shield 20. The cam mechanism 40 includes the upper track 42 having a top section 44 and the lower track 46 having an intermediate section 48 and a bottom section 50. The upper track 42 and the lower track 46 are preferably offset and separately disposed in the upper portion 22 and lower portion 24, respectively, to advantageously improve moldability and to avoid side action during the molding process as described above. The upper track 42 and the lower track 46 include ledges or rails to aid the travel of the knob 70 in the cam mechanism 40.

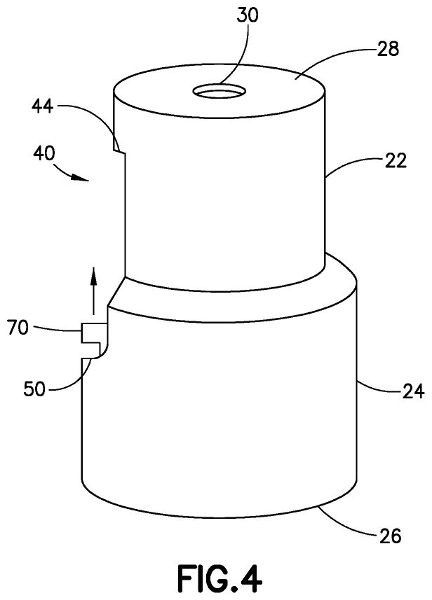

As illustrated in FIG. 4, the cam mechanism 40 provides the opening through the outer surface of the movable shield 20 for a knob 70 of the hub 60 to extend into. The pen needle shield assembly 10 preferably includes two cam mechanisms 40 disposed 180° apart on opposing respective portions of an outer surface of the movable shield 20. However, the pen needle shield assembly 10 can operate sufficiently with one cam mechanism 40. The pen needle shield assembly 10 can also include more than two cam mechanisms 40, if desired, with the understanding that the product design ensures that there is sufficient space.

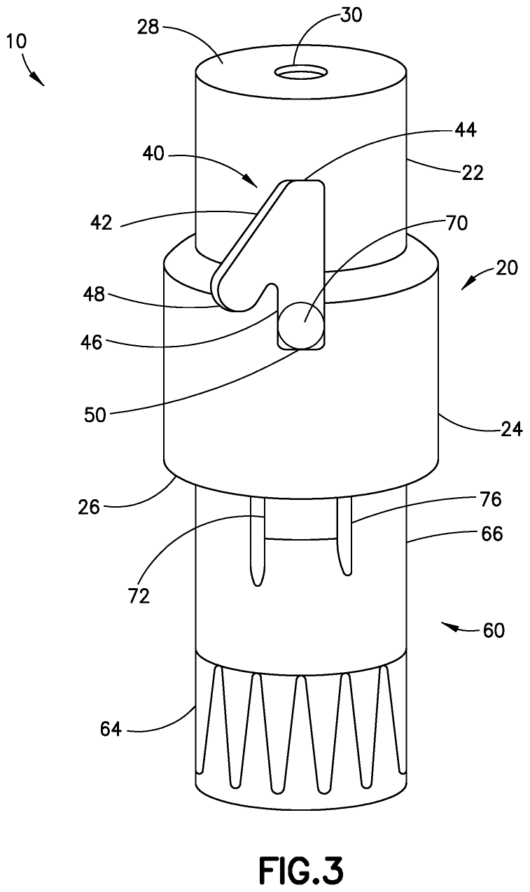

The upper track 42 is disposed in the upper portion 22 of the movable shield 20. The upper track 42 provides a travel path for the knob 70 of the hub 60 to move from a first position where the knob 70 is in the intermediate section 48 to a second position where the knob 70 is in the top section 44. The travel path between the intermediate section 48 and the top section 44 of the upper track 42 is preferably a 45° angled incline with respect to a centerline of the movable shield 20, as illustrated in FIGS. 2 and 3. This travel path exposes the needle cannula 62 and rotates the movable shield 20. The angled incline of the upper track 42 can be defined in a variety of angles as understood by one skilled in the art. FIG. 1 also illustrates a vertical portion of the upper track 42 to prevent rotation of the movable shield 20 when the knob 70 is in the top section 44.

The lower track 46 is disposed in the lower portion 24 of the movable shield 20. The lower portion 24 preferably includes the intermediate section 48 and the bottom section 50 for improved moldability. As illustrated in FIGS. 1-4, the bottom section 50 provides a third position for the knob 70. The bottom section 50 and the intermediate position 48 are disposed in different portions of the lower track 46. Specifically, the knob 70, after use of the needle cannula 62, moves from the second position where the knob 70 is in the top section 44 to the third position where the knob 70 is in the bottom section 50. The travel path between the top section 44 and the bottom section 50 of the cam mechanism 40 is linear or parallel to the centerline of the movable shield 20. However, other an angled travel path between the top section 44 and the bottom section 50 is also contemplated as understood by one skilled in the art. Further details of the operation of the pen needle shield assembly 10 is described below.

The movable shield 20 further includes one or more ramps (not shown) disposed on an inner diametric surface of the lower portion 24. An inner diameter of the one or more ramps gradually decreases in a distal direction. During assembly, the movable shield 20 is pushed toward the hub 60. The one or more ramps advantageously aids to assemble the knob 70 of the hub 60 into the first position where the knob 70 is disposed in the intermediate section 48. The one or more ramps also advantageously prevents the knob 70 from disengaging the intermediate section 48 after assembly.

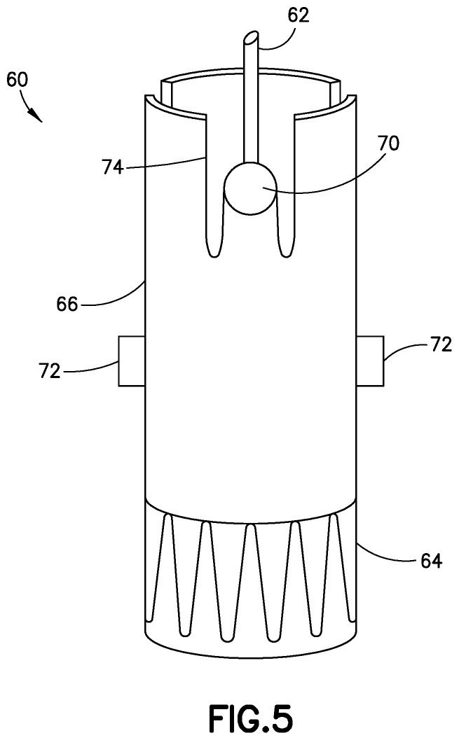

The hub 60 of the pen needle assembly 10 includes the needle cannula 62, a base 64, a body 66, an inner stepped surface 68, a knob 70, an activation stop 72, a first slot 74 and a second slot 76. FIGS. 5 and 6 illustrate that the needle cannula 62 is fixed to a base 64 of the hub 60 as conventionally understood by one skilled in the art. Preferably, a proximal end of the base 64 includes a bore whereby a proximal end of the needle cannula 62 extends within. The bore includes internal threads, for example, that are configured to engage threads of a medication delivery pen or a pen injector, for example. In another embodiment, the proximal end of the needle cannula 62 extends into the bore and beyond the proximal end of the base 64.

A distal end of the needle cannula 62 is exposed in the second position of the pen needle shield assembly 10 whereby the needle cannula 62 extends out of the distal opening 30. On the other hand, the distal end of the needle cannula 62 is unexposed in the first and third positions of the pen needle shield assembly 10 whereby the needle cannula 62 is retracted and shielded in the movable shield 20.

FIGS. 5 and 6 further illustrate that the body 64 of the hub 60 includes the inner stepped surface 68, the knob 70, the activation stop 72, the first slot 74 and the second slot 76. The inner stepped surface 68 includes a ledge on an inner diameter of the body 64 to provide a contact surface for a proximal end of the spring 80. When the pen needle shield assembly 10 is assembled, a distal end of the spring 80 contacts an inner surface (not shown) adjacent to the distal end 28 of the movable shield 20. The spring 80 is preferably a coil spring. Operation of the spring 80 in the pen needle shield assembly 10 will be further described below.

The first slot 74 of the hub 60 extends from the distal end of the body 66 of the hub 60 to approximately a central portion of the body 66. The first slot 74 is wider than the width of the knob 70 so that a cantilever effect is experienced. Specifically, the knob 70 is disposed on an outer surface of the body 64 of the hub 60 near its distal end. The knob 70 is disposed on a flanged member that extends into the first slot 74 of the hub 60 in a cantilever manner. Accordingly, the flanged member provides elastic movement of the knob 70 during assembly of the hub 60 to the movable shield 20. Specifically, the flanged member of the knob 70 compresses during assembly and elastically returns to its natural state when the knob 70 is engaged to the cam mechanism 40.

As described above, when assembled, the knob 70 cooperates with the upper track 42 and the lower track 46 of the cam mechanism 40. The knob 70 extends outwardly from an outer surface of the body 64 of the hub 60. Preferably, two knobs 70 are respectively disposed on opposing portions of an outer surface of the body 64 of the hub 60. However, one knob 70 is sufficient for proper operation of the pen needle shield assembly 10.

The second slot 76 of the body 66 of the hub 60 extends from the distal end of the body 66 to a portion adjacent to the proximal end of the body 66. The second slot 66 is wider than the width of the activation stop 72 so that a cantilever effect is experienced. Specifically, the locking mechanism includes the activation stop 72 of the hub 60 that cooperates with the proximal end 26 of the movable shield 20. The activation stop 72 is disposed on a flanged member extending into the second slot 76 of the body 66 of the hub 60. The flanged member provides elastic movement during operation of the pen needle shield assembly 10. The activation stop 72 is disposed near the proximal end of the body 66 of the hub 60 and extends outwardly from the flanged member.

The activation stop 72 is positioned distally from the knob 70. Preferably, two activation stops 72 are respectively disposed 180° apart on opposing portions of an outer surface of the body 64 of the hub 60. However, one activation stop 72 is sufficient for proper operation of the pen needle shield assembly 10. The pen needle shield assembly 10 can also include more than two activation stops 72, if desired, with the understanding that the product design ensures that there is sufficient space.

FIG. 2 illustrates that when the pen needle shield assembly 10 is disposed in the first and second positions as described above, the activation stop 72 is compressed and surrounded by a proximal portion of the movable shield 20. FIG. 3 illustrates that when the pen needle shield assembly 10 is disposed in the third position, the activation stop 72 is released from the movable shield 20 and is no longer compressed. Accordingly, the activation stop 72 elastically moves outward and returns to its original form.

In the third position, the activation stop 72 subsequently contacts the proximal end 26 of the movable shield 20 to prevent movement of the movable shield 20 toward the hub 60. In this position, the bottom section 50 of the cam mechanism 40 prevents the movable shield 20 from moving away from the hub 60. Thus, the locking mechanism is activated to ensure single use of the needle cannula 62 and to avoid inadvertent needle use or contact.

One or more ramps (not shown) can also be disposed near the proximal end 26 on an inner diameter of the lower portion 24 for engaging the activation stop 72. The ramp can provide a smooth movement of the activation stop 72 in the movable shield 20 between the first and second positions and as the knob 70 moves to the third position. As described above, once the knob 70 is in the third position, the activation stop 72 is no longer compressed by the movable shield 20, elastically extends outwardly and establishes the locking mechanism between the activation stop 72 and the proximal end 26 of the movable shield 20.

Assembly and operation of the pen needle shield assembly 10 is described below. For assembly, the spring 80 is first disposed in the hub 60 as illustrated in FIG. 6. Specifically, the proximal end of the spring 80 rests on the inner stepped surface 68 of the hub 60.

Next, the hub 60 is assembled to the movable shield 20 and snap fit. In particular, the knob 70 is initially depressed to position the knob 70 under the outer surface of the lower portion 24 of the movable shield 20. As illustrated in FIGS. 1, 2 and 4, the knob 70 moves toward the cam mechanism 40 and traverses the ramp (not shown) of the movable shield 20. The assembly is complete when the knob 70 ultimately is aligned to and engages the intermediate section 48 of the cam mechanism 40 to establish the snap fit. The ramp elastically compresses the knob 70 in a gradual manner until the knob 70 engages the intermediate section 48. After the knob 70 is engaged to the intermediate section 48, and assembly is complete, the knob 70 elastically returns to its original form. Accordingly, the ramp prevents the knob 70 from disengaging the cam mechanism 40, thus maintaining engagement between the movable shield 20 and the hub 60.

At the same time, as illustrated in FIG. 2, the activation stop 72 is compressed during assembly and disposed underneath the outer wall of the lower portion 24 near the proximal end 26 of the movable shield 20. In this first position, the spring 80 is compressed between the inner stepped surface 68 and the inner wall (not shown) adjacent to the distal end 28 of the movable shield 20. The compression of the spring in the first position applies a force to keep the knob 70 in the first position. Also, as illustrated in FIGS. 1 and 2, the needle cannula 62 is retracted in the movable shield 20 in the first position.

Operation of the pen needle shield assembly 10 is now described below. First, a clinician or patient mounts the pen needle shield assembly 10 onto the medication delivery pen or the fluid injector, for example. Specifically, the base 64 includes internal threads that engage threads of the medication delivery or the fluid injector. When the clinician or user is ready for medication delivery, the clinician or user depresses the pen needle shield assembly 10 onto a skin surface. This causes the movable shield 20 to move toward the hub 60 and rotate as the knob 70 travels along the upper track 42 of the cam mechanism 40 to the second position.

In the second position, the knob 70 is disposed in the top section 44 and the needled cannula 62 extends through the distal opening 30 and enters into the skin surface of the patient for medication delivery. FIGS. 1 and 2 illustrate the path the knob 70 travels to move from the first position to the second position. When the knob 70 moves from the first position to the second position, the activation stop 72 moves distally with respect to the proximal end 26 of the movable shield 20. In the second position, the spring 80 is further compressed.

When medication delivery is complete, the user or clinician removes the needle cannula 62 and the pen needle shield assembly 10 from the skin surface of the patient. This causes the spring 80 to release and apply a force to push the movable shield 20 away from the hub 60. As a result, as illustrated in FIG. 3, the needle cannula 62 is again shielded by the movable shield 20. Also, the knob 70 moves from the second position at the top section 44 to the third position at the bottom section 50 of the cam mechanism 40.

This movement advantageously provides a pen needle such as a safety pen needle with a passive needle stick prevention feature such that the clinician or patient does not have to actively cover the distal end of the needle cannula 62. Instead, the pen needle shield assembly 10 automatically shields the needle cannula 62 after use via the applied force from the spring 80.

The movable shield 20 does not automatically rotate when traveling from the second position to the third position. However, the user or clinician can undesirably tamper with the pen needle shield assembly 10 and rotate the movable shield 20 while the knob 70 is moving from the second position to the third position and before the locking mechanism is activated.

The bottom section 50 is proximal to the intermediate section 48. The bottom section 50 is advantageously positioned so that the activation stop 72 is no longer disposed under the lower portion 24 of the movable shield 20. Instead, in the third position, the activation stop 72 elastically moves outward and engages the proximal end 26 of the movable shield 20.

In the third position, the locking mechanism is established between the movable shield 20 and the activation stop 72 to provide needle stick prevention and to ensure single use of the needle cannula 62. Specifically, the movable shield 20 is not able to move away from the hub 60 because the knob 70 is engaged to the lower track 46 at the bottom section 50. The movable shield 20 is not able to further compress the spring 80 and move toward the hub 60 because the activation stop 72 of the hub 60 contacts the proximal end 26 of the movable shield 20.

The foregoing detailed description of the certain exemplary embodiments has been provided for the purpose of explaining the principles of the invention and its practical application, thereby enabling others skilled in the art to understand the invention for various embodiments and with various modifications as are suited to the particular use contemplated. This description is not necessarily intended to be exhaustive or to limit the invention to the precise embodiments disclosed. Any of the embodiments and/or elements disclosed herein may be combined with one another to form various additional embodiments not specifically disclosed, as long as they do not contradict each other. Accordingly, additional embodiments are possible and are intended to be encompassed within this specification and the scope of the invention. The specification describes specific examples to accomplish a more general goal that may be accomplished in another way.

As used in this application, the terms “front,” “rear,” “upper,” “lower,” “upwardly,” “downwardly,” and other orientational descriptors are intended to facilitate the description of the exemplary embodiments of the present invention, and are not intended to limit the structure of the exemplary embodiments of the present invention to any particular position or orientation. Terms of degree, such as “substantially” or “approximately” are understood by those of ordinary skill to refer to reasonable ranges outside of the given value, for example, general tolerances associated with manufacturing, assembly, and use of the described embodiments.

What is claimed is:

1. A pen needle shield assembly comprising:
a hub fixed to a needle cannula; and
a movable shield surrounding the hub, the movable shield comprising:
an upper portion having an outer diameter;
a lower portion having an outer diameter greater than the outer diameter of the upper portion; and
a distal opening; wherein
the movable shield moves from a first position where the needle cannula is retracted in the movable shield prior to use, to a second position where the needle cannula is extended out of the movable shield through the distal opening for medication delivery and to a third position where the needle cannula is retracted in the movable shield and locked to prevent further use.

2. The pen needle shield assembly of claim 1, wherein:
the movable shield includes a cam mechanism having an upper track and a lower track;
the upper track of the cam mechanism is disposed in the upper portion; and
the lower track of the cam mechanism is disposed in the lower portion.

3. The pen needle shield assembly of claim 2, wherein
the lower track of the cam mechanism includes an intermediate section corresponding to the first position and a bottom section corresponding to the third position; and
the third position is proximal to the first position.

4. The pen needle shield assembly of claim 2, wherein the hub includes a knob that comprises a flexible flange that extends outwardly and is configured to engage the cam mechanism.

5. The pen needle shield assembly of claim 4, wherein the knob includes two knobs disposed 180 degrees from each other.

6. The pen needle shield assembly of claim 4, wherein
the cam mechanism includes an opening that extends through an outer wall of the movable shield; and
the knob extends into the slot of the cam mechanism.

7. The pen needle shield assembly of claim 4, wherein the knob engages the lower track in the first position, engages the upper track in the second position and engages the lower track in the third position.

8. The pen needle shield assembly of claim 7, wherein the knob in the first position and in the third position engage different portions of the lower track.

9. The pen needle shield assembly of claim 1, wherein the movable shield rotates when moving from the first position to the second position.

10. The pen needle shield assembly of claim 9, wherein the movable shield does not rotate when moving from the second position to the third position.

11. The pen needle shield assembly of claim 1, wherein the hub includes an activation stop that locks the movable shield in the third position.

12. The pen needle shield assembly of claim 11, wherein the activation stop includes two activation stops disposed 180 degrees from each other.

13. The pen needle shield assembly of claim 11, wherein the activation stop comprises a flexible flange that is elastically compressed in the first and second position and is released in the third position to extend outwardly and contact a proximal end of the movable shield.

14. The pen needle shield assembly of claim 1, further comprising a coil spring that is compressed during use of the pen needle shield assembly in the second position.

15. The pen needle shield assembly of claim 14, wherein the coil spring applies a force to keep the movable shield in the first position prior to use.

16. The pen needle shield assembly of claim 14, wherein the coil spring applies a force to move the movable shield from the second position to the third position.

17. The pen needle shield assembly of claim 14, wherein
the hub includes an inner stepped surface; and
the spring contacts the inner stepped surface of the hub and an inner surface adjacent to a distal end of the movable shield.
